(19) United States
(12) Patent Application Publication
(10) Pub. No.: US 2017/0333632 A1
(43) Pub. Date: Nov. 23, 2017

(54) Multi-Chamber Syringes And Methods For Using The Same

(71) Applicants: Olivia Arrendondo, West Sacramento, CA (US); Kirsten McGowan, Redwood City, CA (US); Elizabeth Riemann, Spring, TX (US); Jacalyn Rogers, Chardon, OH (US); Mona D. Shah, Pearland, TX (US); Tracey A. Stoll, San Jose, CA (US); Sarah Trowbridge, Davis, CA (US); Jeffrey S. Hovis, Boston, MA (US)

(72) Inventors: Olivia Arrendondo, West Sacramento, CA (US); Kirsten McGowan, Redwood City, CA (US); Elizabeth Riemann, Spring, TX (US); Jacalyn Rogers, Chardon, OH (US); Mona D. Shah, Pearland, TX (US); Tracey A. Stoll, San Jose, CA (US); Sarah Trowbridge, Davis, CA (US); Jeffrey S. Hovis, Boston, MA (US)

(21) Appl. No.: 15/603,117

(22) Filed: May 23, 2017

Related U.S. Application Data
(60) Provisional application No. 62/339,994, filed on May 23, 2016.

(51) Int. Cl. A61M 5/19; A61M 5/31; A61M 5/315; A61M 5/34; A61M 5/178

(52) U.S. Cl. CPC A61M 5/19; A61M 5/31596; A61M 5/34; A61M 5/3129; A61M 2005/1787; A61M 2005/3143; A61M 2205/19; A61M 2210/12; A61M 2005/3128

(57) ABSTRACT

A multi-chamber syringe for delivering a plurality of fluids to a central venous catheter includes a barrel and a plunger. The barrel includes a plurality of chambers, and a first one of the plurality of chambers has a predefined volume of a saline flush stored therein. Actuation of the plunger causes the predefined volume of the saline flush to be dispensed from the barrel into the central venous catheter.

### CROSS-REFERENCE TO RELATED APPLICATIONS

This application claims the benefit of and priority to U.S. Provisional Application No. 62/339,994, filed on May 23, 2016, which is hereby incorporated by reference herein in its entirety.

### FIELD OF THE PRESENT DISCLOSURE

The present disclosure relates generally to multi-chamber syringes, and more particularly, to multi-chamber syringes wherein at least one chamber is prefilled with a flushing solution.

### BACKGROUND

Syringes are used worldwide in a multitude of healthcare settings. Medical syringes are generally used to deliver a fluid (e.g., medication) to a patient or draw a sample of fluid (e.g., blood) from the patient. For example, the tip of the syringe may be coupled to a needle via a Luer lock for directly puncturing a patient's skin in order to deliver medication directly into the bloodstream. In addition, catheters are often used to deliver fluid between the syringe and the patient. For instance, the syringe can be coupled to an intravenous catheter (“IV”) or a central venous catheter (commonly referred to as a “central line”).

Often, effective patient treatment requires sequential delivery of two or more different fluids into the patient's bloodstream, thereby requiring two or more syringes. For instance, a first syringe is coupled to the central line to deliver medication, and a second syringe is coupled to the central line to deliver a flushing solution. Using multiple syringes in a single patient encounter gives rise to a number of difficulties and inefficiencies. As an initial matter, the medical facility or provider must store and maintain a large supply of syringes. To illustrate this difficulty, a central venous catheter is often accessed up to 75 times per day per patient, requiring as many as 150 syringes per patient per day. Further, the medical facility or provider must then also safely dispose of these used syringes. There are also patient-associated risks involved in the use of multiple syringes. For example, because a healthcare provider (i.e., nurse or technician) must manually actuate the syringe to draw a fluid (e.g., a medication) into the syringe and/or to deliver the fluid, and multiplying these steps increases the likelihood of human error. Further, each use of a syringe carries the risk of contamination, and thus the risk of patient infection in a given procedure is directly correlated with the number of syringes that are used. Central line-associated bloodstream infections (“CLABSI”), which are often caused by contaminated syringes, result in thousands of deaths and billions of dollars in added costs per year in the United States alone. Thus, new devices and methods for sequentially delivering a plurality of fluids from a single syringe are needed. The present disclosure is directed towards addressing these needs and other problems.

### SUMMARY OF THE PRESENT DISCLOSURE

According to some implementations of the present disclosure, a multi-chamber syringe for delivering a plurality of fluids to a central venous catheter includes a barrel and a plunger. The barrel includes a plurality of chambers, and a first one of the plurality of chambers has a predefined volume of a saline flush stored therein. Actuation of the plunger causes the predefined volume of the saline flush to be dispensed from the barrel into the central venous catheter.

According to some implementations of the present disclosure, a multi-chamber syringe includes a barrel, a gasket, an end stopper, and a plunger. The barrel includes an upper opening, a central cavity, and a tip having a lower opening. The gasket and the end stopper are disposed within the central cavity such that (i) a first chamber of the central cavity is defined by a space between the tip and the gasket, and (ii) a second chamber of the central cavity is defined by a space between the gasket and the end stopper. The gasket includes a valve assembly configured to move between a closed position and an open position. The first chamber has a predefined volume of flushing solution stored therein. The plunger is slidably disposed within the central cavity such that the plunger is moveable with respect to the barrel. Responsive to the valve assembly of the gasket being in the open position, actuation of the plunger causes at least a portion of the predefined volume of flushing solution stored in the first chamber to be dispensed through the lower opening of the tip.

According to some implementations of the present disclosure, a method for delivering a fluid to a central venous catheter includes providing a multi-chamber syringe having a barrel including an upper opening, a central cavity, and a tip having a lower opening. The multi-chamber syringe further includes a gasket having a valve assembly and an end stopper disposed within the central cavity such that (i) a first chamber of the central cavity is defined by a space between the gasket and the end stopper, and (ii) a second chamber of the central cavity is defined by a space between the tip and the gasket, the first chamber having a predefined volume of flushing solution stored therein and the second chamber having a predefined volume of fluid stored therein. The multi-chamber syringe also includes a plunger slidably disposed within the central cavity. The tip of the multi-chamber syringe is coupled to an access point of the central venous catheter and the plunger is moved from a first position to a second position such that the predefined volume of fluid stored in the second chamber is dispensed through the lower opening of the tip. The plunger is moved from the second position to a third position such that the predefined volume of flushing solution stored in the first chamber is dispensed through the lower opening of the tip through the second chamber and the valve assembly of the gasket.

The present disclosure is susceptible to various modifications and alternative forms, and some representative implementations have been shown by way of example in the drawings and will be described in detail herein. It should be understood, however, that the inventive aspects of the disclosure are not limited to the particular forms disclosed. Rather, the disclosure is to cover all modifications, equivalents, and alternatives falling within the spirit and scope of the present disclosure as defined by the appended claims.

### BRIEF DESCRIPTION OF THE DRAWINGS

FIG. 1 is a schematic illustration of a multi-chamber syringe according to some implementations of the present disclosure;

FIG. 2 is a cross-sectional side view of a multi-chamber syringe according to some implementations of the present disclosure;

FIG. 3A is a cross-sectional side view of a first gasket of the multi-chamber syringe of FIG. 2 having a first valve assembly in a closed position according to some implementations of the present disclosure;

FIG. 3B is a cross-sectional side view of a second gasket of the multi-chamber syringe of FIG. 2 having a second valve assembly in a closed position according to some implementations of the present disclosure;

FIG. 4A is a cross-sectional side view of the first gasket of FIG. 3A with the first valve assembly in an open position according to some implementations of the present disclosure; and

FIG. 4B is a cross-sectional side view of a second gasket of FIG. 3B with the second valve assembly in an open position according to some implementations of the present disclosure;

While the present disclosure is susceptible to various modifications and alternative forms, specific embodiments and implementations are shown by way of example in the drawings and are described in detail herein. It should be understood, however, that the present disclosure is not intended to be limited to the particular forms disclosed. Rather, the present disclosure is to cover all modifications, equivalents, and alternatives falling within the spirit and scope of the present disclosure.

### DETAILED DESCRIPTION

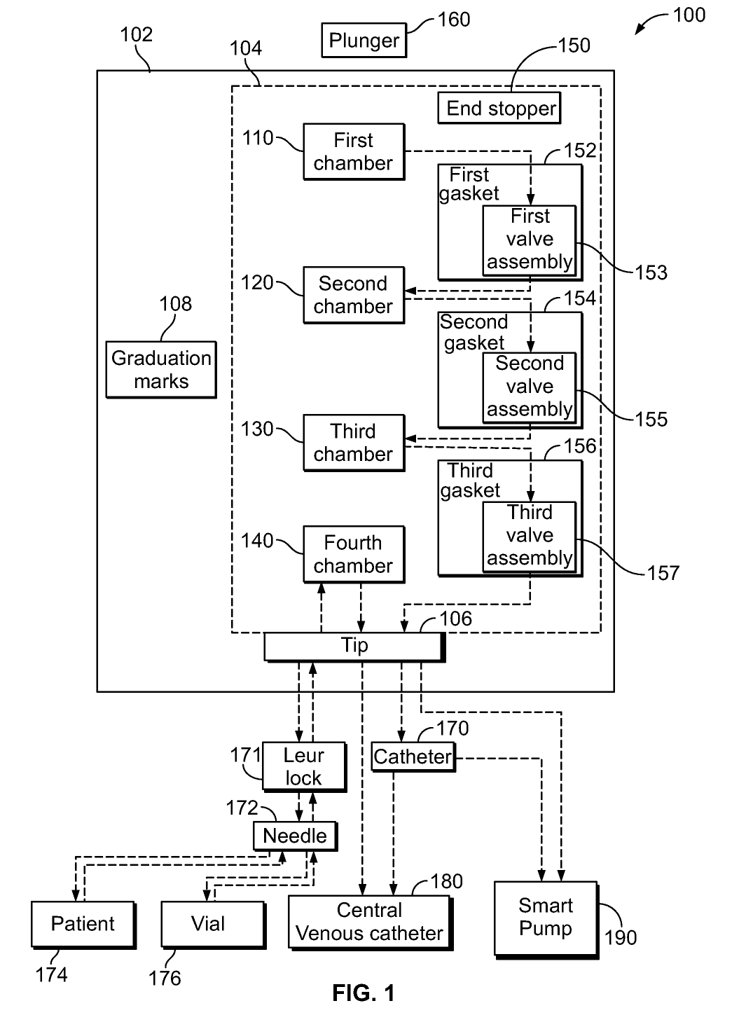

Referring to FIG. 1, a multi-chamber syringe 100 includes a barrel 102, a first gasket 152, a second gasket 154, a third gasket 156, an end stopper 150, and a plunger 160. The multi-chamber syringe 100 is generally used to sequentially deliver a plurality of fluids stored in the barrel 102 to a patient (e.g., via a central venous catheter) and/or draw one or more fluids into the barrel 102 for storage.

The barrel 102 includes a central cavity 104, a tip 106, and graduation marks 108. The barrel 102 can be made from a glass material, a medical-grade polymer material, or the like, or any combination thereof. The central cavity 104 of the barrel 102 is a hollow cavity or aperture having a volume ranging between about 0.5 mL and about 60 mL for storing a volume of fluid within the barrel 102. The central cavity 104 is in fluid communication with the tip 106 such that fluid stored in the central cavity 104 can flow out of the barrel 102 through the tip 106, and vice versa.

The graduation marks 108 are displayed on an outer surface of the barrel 102, (which is made from a transparent or semi-transparent material) and permit a user to determine the volume of fluid stored in the central cavity 104 of the barrel 102 at a given time. For instance, if the central cavity 104 of the barrel 102 has a capacity of about 10 mL, the graduation marks 108 can include at least ten incremental indications of volume (i.e., 1 mL, 2 mL, 3 mL, 4 mL, etc). The graduation marks 108 can be directly printed on the barrel 102 or coupled to the outer surface (e.g., using an adhesive connection). Further, the graduation marks 108 can be oriented on the outer surface of the barrel 102 such that they are readable in one direction (i.e., as fluid is being dispensed from the barrel 102), or in two directions (i.e., as fluid is being dispensed from the barrel 102 and as fluid is being drawn into the barrel 102).

The end stopper 150, the first gasket 152, the second gasket 154, and the third gasket 156 are disposed within the central cavity 104 of the barrel 102. Each of the gaskets 152, 154, and 156 are spaced relative to one another within the central cavity 104 such that (i) a space between the tip 106 and the first gasket 152 defines a first chamber 110, (ii) a space between the first gasket 152 and the second gasket 154 defines a second chamber 120, (iii) a space between the second gasket 154 and the third gasket 156 defines a third chamber 130, and (iv) a space between the third gasket 156 and the end stopper 150 defines a fourth chamber 140. In this manner, the central cavity 104 can be divided into a plurality of chambers depending on the number of gaskets disposed within the central cavity 104 (e.g., four gaskets define five chambers, three gaskets define four chambers, two gaskets define three chambers, one gasket defines one chamber, etc.)

Each of the gaskets 152, 154, 156 and the end stopper 150 are made from a polymer material (e.g., rubber) and are sized and shaped such that each gasket creates an air-tight seal within the central cavity 104. For example, the first gasket 152 prevents fluid communication between the first chamber 110 and the second chamber 120. In this manner, four separate fluids can be stored in the multi-chamber syringe 100 without any mixing between the fluids. To permit fluid to flow between adjacent chambers (e.g., from the second chamber 120 to the first chamber 110), the first gasket 152 includes a first valve assembly 153, the second gasket includes a second valve assembly 155, and the third gasket includes a third valve assembly 157. Each of the valve assemblies 153, 155, and 157 has a closed position and an open position for permitting fluid to pass through the respective gasket. For example, when the first valve assembly 153 is in the closed position, fluid stored in the second chamber 120 cannot flow into the first chamber 110 because the first valve assembly 153 and the first gasket 152 form a seal within the central cavity 104. When the first valve assembly 153 is in the open position, fluid stored in the second chamber 120 can pass through the first gasket 152 through the first valve assembly 153 and into the first chamber 110.

The plunger 160 is partially disposed within the central cavity 104 of the barrel 102 such that it is moveable relative to the barrel 102. Actuation of the plunger 160 towards the tip 106 causes fluid to be dispensed from the barrel 102 through the tip 106. For example, in some implementations, a first fluid is stored in the first chamber 110, a second fluid is stored in the second chamber 120, a third fluid is stored in the third chamber 130, and a fourth fluid is stored in the fourth chamber 140. In such implementations, actuation of the plunger 160 (i.e., moving the plunger 160 from a first position towards a second position) causes at least a portion of the first fluid to be dispensed from the first chamber 110 through the tip 106. Once the first fluid is completely dispensed from the first chamber 110 (i.e., the plunger 160 is in the second position), the first gasket 152 contacts the bottom of the central cavity 104 (i.e., at or near the tip 106), which moves the first valve assembly 153 from the closed position to the open position. The first gasket 152 will only contact the bottom of the central cavity 104 when the first fluid is completely dispensed because the first fluid is substantially incompressible. With the first valve assembly 153 in the open position, actuation of the plunger 160 from the second position towards a third position causes at least a portion of the second fluid stored in the second chamber 120 to pass through the first valve assembly 153 and be dispensed from the central cavity 104 through the tip 106. Once the second fluid is completely dispensed from the second chamber 120 (i.e, the plunger 160 is in the third position), the second gasket 154 contacts the first gasket 152 and moves the second valve assembly 155 from the closed position to the open position. With the second valve assembly 155 of the second gasket 154 in the open position, actuation of the plunger 160 causes at least a portion of the third fluid stored in the third chamber 130 to be dispensed through the second valve assembly 155, the first valve assembly 153, and the tip 106. Once the third fluid is completely dispensed from the third chamber 130 (i.e., the plunger 160 is in the third position), the third gasket 156 moves down towards the tip 106 and contacts the second gasket 154, moving the third valve assembly 157 from the closed position to the open position. Once the third valve assembly 157 is in the open position, actuation of the plunger 160 from the third position towards a forth position causes at least a portion of the fourth fluid stored in the fourth chamber 140 to be dispensed through the third valve assembly 157, the second valve assembly 155, the first valve assembly 153, and the tip 106. In this manner, the first fluid, second fluid, third fluid, and fourth fluid can be sequentially dispensed from the multi-chamber syringe 100.

The tip 106 can be coupled to various external components to deliver fluid(s) stored in the central cavity 104 of the barrel 102 (e.g., the first chamber 110) to a patient, another medical device, and/or to draw fluid(s) into the central cavity 104. In some implementations, the tip 106 is coupled to a Luer lock 171, which is in turn coupled to a needle 172 such that fluid dispensed from the tip 106 flows through the needle 172. The Luer lock 171 can be coupled to the tip 106 by various fastening mechanisms, such as, for example, a threaded connection, an adhesive connection, a friction-fit connection, or the like, or any combination thereof. Alternatively, the Luer lock 171 and the tip 106 can be unitary and/or monolithic. A proximal end of the needle 172 is secured within a central aperture of the Luer lock 171 and a distal end of the needle 172 has a sharp edge or point for penetrating the skin of a patient 174 or the seal of a vial 176 (e.g., a vial containing a medication). The proximal end of the needle 172 can be coupled to the Luer lock 171 using various fastening mechanisms, such as, for example, a threaded connection, an adhesive connection, a friction-fit connection, a snap-fit connection, or the like, or any combination thereof.

In some implementations, the tip 106 can be coupled to a catheter 170. The catheter 170 is a generally cylindrical hollow tube made from a medical-grade polymer material that is commonly used in a variety of healthcare settings. A proximal end of the catheter 170 is coupled to the tip 106 of the barrel 102, and a distal end of the catheter 170 is coupled to another medical apparatus (e.g., another catheter). The distal end of the catheter 170 can be coupled to the tip 106 using various fastening mechanisms, such as, for example, a threaded connection, an adhesive connection, a friction-fit connection, a snap-fit connection, or the like, or any combination thereof. Advantageously, the catheter 170 is flexible and has a greater length than the needle 172 to permit delivery of fluid from the multi-chamber syringe 100 over greater distances.

In some implementations, the distal end of the catheter 170 is coupled to an access port (commonly referred to as a “hub”) of a central venous catheter 180. The central venous catheter 180 is a generally cylindrical hollow tube that is often placed in a large vein of a patient to deliver medication or fluids, or to collect blood for medical testing. The central venous catheter 180 is different from an intravenous catheter (“IV”) in that central venous catheters are placed close to the patient's heart and can remain in place for weeks or months at a time. When connected to the central venous catheter 180 via the catheter 170, the multi-chamber syringe 100 can be used to sequentially deliver fluids to the central venous catheter 180 via actuation of the plunger 160 in the manner described above. Alternatively, the tip 106 of the barrel 102 can be directly coupled to the access port of the central venous catheter 180.

In other implementations, the catheter 170 or the tip 106 of the barrel 102 of the multi-chamber syringe 100 can be coupled to an access port of a smart pump 190. The smart pump 190, in turn, can be coupled to another medical apparatus such as, for example, the central venous catheter 180. The smart pump 190 delivers a precise volume of fluid (e.g., to the central venous catheter 180) according to a programmable delivery rate and duration entered by a user. In such implementations, the smart pump 190 controls the rate at which the plunger 160 of the multi-chamber syringe 100 is actuated (i.e., moved relative to the barrel 102 towards or away from the tip 106). Thus, the smart pump 190 can be used to sequentially deliver various fluids from the multi-chamber syringe 100 at a predefined rate and/or for a predefined duration.

Various fluids can be stored in the first chamber 110, the second chamber 120, the third chamber 130, and/or the fourth chamber 140. For example, a flushing solution can be stored in one or more of the four chambers. The flushing solution is generally used to keep lines (e.g., the central venous catheter 180) and access ports clear and sterile, to ensure that a medication is fully delivered, and/or to ensure that two different medications do not adversely react with one another. The flushing solution can be, for example, a saline flush, a heparin flush, an antibiotic flush, or any combination thereof. The saline flush is a sterile mixture of salt and water that can be used for any of the functions described above. The heparin flush (often referred to as a “heparinised saline”) is an anticoagulant that is generally used to prevent clotting and blockage (e.g., in the central venous catheter 180). The antibiotic flush is generally used to eliminate any contamination in the lines (e.g., in the central venous catheter 180). Other fluids having healthcare applications can also be stored in the multi-chamber syringe. For example, a medication, a nutritional fluid, a supplement fluid, or any combination thereof can be stored in one or more the first chamber 110, the second chamber 120, the third chamber 130, and/or the fourth chamber 140.

In some implementations, a plurality of fluids can be stored in the multi-chamber syringe 100 so that the multi-chamber syringe 100 can be used for a saline-administer-saline-heparin (“SASH”) protocol. In such implementations, a first saline flush is stored in the first chamber 110, a medication, a nutritional fluid, and/or a supplement fluid is stored in the second chamber 120, a second saline flush is stored in the third chamber 130, and a heparin flush is stored in the fourth chamber 140. Actuation of the plunger 160 first causes the four fluids to be sequentially dispensed through the tip 106: the first saline flush is dispensed first, the medication, nutritional fluid, and/or supplement fluid is dispensed second, the second saline flush is dispensed third, and the heparin flush is dispensed fourth. The first saline flush clears the line (e.g. the catheter 170 and/or the central venous catheter 180) prior to the administration of the medication, nutritional, and/or supplement fluid. The second saline flush ensures that all of the medication, nutritional, and/or is administered to the patient, and the heparin flush (i.e., anticoagulant) decreases blood clotting to increase the clinical effectiveness of the medication, nutritional fluid, and/or supplement fluid.

In other implementations, the multi-chamber syringe 100 can be configured for a saline-administer-saline-antibiotic (“SASA”) protocol. A first saline flush is stored in the first chamber 110, a medication, a nutritional fluid, and/or a supplement fluid is stored in the second chamber 120, a second saline flush is stored in the third chamber 130, and an antibiotic flush is stored in the fourth chamber 140. In this configuration, actuation of the plunger 160 first causes the four fluids to be sequentially dispensed through the tip 106: the first saline flush is dispensed first, the medication, nutritional fluid, and/or supplement fluid is dispensed second, the second saline flush is dispensed third, and the antibiotic flush is dispensed fourth. The first saline flush clears the line (e.g. the catheter 170 and/or the central venous catheter 180) prior to the administration of the medication, nutritional, and/or supplement fluid. The second saline flush ensures that all of the medication, nutritional, and/or is administered to the patient, and the antibiotic flush sterilizes the line (e.g., central venous catheter 180) and decreases the likelihood of infection in the patient. While the multi-chamber syringe 100 is shown as including all of the components described above, more or fewer components can be included in a multi-chamber syringe. For example, an alternative multi-chamber syringe (not shown) includes the barrel 102, the end stopper 150, the plunger 160, and the first gasket 152 (i.e., such that the central cavity 104 includes the first chamber 110 and the second chamber 120, but not the third chamber 130 or the fourth chamber 140). As another example, a second alternative multi-chamber syringe (not shown) includes the barrel 102, the plunger 160, the end stopper 150, the first gasket 152, and the second gasket 154 (i.e., such that the central cavity 104 includes the first chamber 110, the second chamber 120, and the third chamber 130, but not the fourth chamber 140). Thus, various multi-chamber syringes can be formed using any portion of the basic components described herein.

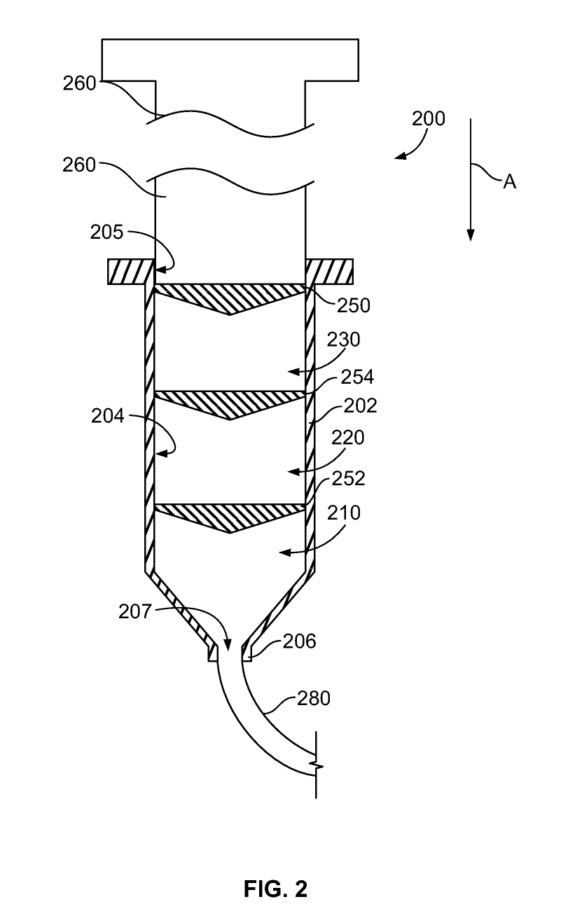

Referring to FIG. 2, a multi-chamber syringe 200 that is similar to the multi-chamber syringe 100 (as indicated by like reference numbers) includes a barrel 202 and a plunger 260. The barrel 202 includes a central cavity 204, an upper opening 205, a tip 206, and a lower opening 207. The multi-chamber syringe 200 also includes an end stopper 250, a first gasket 252, and a second gasket 254 disposed within the central cavity 204 of the barrel 202.

Like the multi-chamber syringe 100, the first gasket 252, and the second gasket 254, and the end stopper 250 are spaced relative to one another within the central cavity 204 such that (i) a space between the tip 206 and the first gasket 252 defines a first chamber 210, (ii) a space between the first gasket 252 and the second gasket 254 defines a second chamber 220, and (iii) a space between the second gasket 254 and the end stopper 250 defines a third chamber 230. The multi-chamber syringe 200 differs from the multi-chamber syringe 100 in that the central cavity 204 does not include a third gasket and a fourth chamber.

A first fluid is stored in the first chamber 210, a second fluid is stored in the second chamber 220, and a third fluid is stored in the third chamber 230. In some implementations, similar to the SASH and SASA protocols described above, the multi-chamber syringe 200 can be configured for a saline-administer-saline (“SAS”) protocol. In such implementations, the first fluid stored in the first chamber 210 is a first saline flush, the second fluid stored in the second chamber 220 is a medication, a nutritional fluid, and/or a supplement fluid, and the third fluid stored in the third chamber 230 is a second saline flush. Thus, actuation of the plunger 260 causes the first saline flush to be dispensed first, the medication, nutritional fluid, and/or supplement fluids second, and the second saline flush third. In this manner, the multi-chamber syringe 200 can be coupled to a central venous catheter 280 and sequentially deliver the first saline flush, medication, and the second saline flush to a patient as required by the given medication application.

More specifically, as the plunger 260 is moved in the direction of arrow A (i.e., towards the tip 206 of the barrel 202), the plunger 260 contacts and moves the end stopper 250 in the direction of arrow A within the central cavity 204 of the barrel 202. Because the third fluid (e.g., the second saline flush) stored in the third chamber 230 is substantially incompressible, the third fluid causes the second gasket 254 to move in the direction of arrow A as well. Likewise, because the second fluid (e.g., medication) stored in the second chamber 220 is substantially incompressible, the second fluid causes the first gasket 252 to move in the direction of arrow A within the central cavity 204 of the barrel 202. As the first gasket 252 moves towards the tip 206, the first fluid stored in the first chamber 210 (e.g., first saline flush) is dispensed through the tip 206 and the lower opening 207 into, for example, the central venous catheter 280.

Once the first fluid stored in the first chamber 210 is completely dispensed, the first gasket 252 contacts the bottom of the central cavity 204 at or near the tip 206, which moves a first valve assembly (not shown) from a closed position to an open position. As the plunger 260 is then moved in the direction of arrow A, the second fluid stored in the second chamber 220 is dispensed through the first valve assembly and the tip 206 into the central venous catheter 280. Similarly, once the second fluid stored in the second chamber 220 is completely dispensed, the second gasket 254 contacts the first gasket 252, moving a second valve assembly (not shown) from a closed position to an open position. As the plunger 260 is then moved in the direction of arrow A, the third fluid stored in the third chamber 230 is dispensed through the second valve assembly, the first valve assembly, and the tip 206 and into the central venous catheter 280.

To assemble and prefill the multi-chamber syringe 200, the central cavity 204 is filled with a predefined volume of a first fluid (e.g., a first saline flush), and the first gasket 252 is inserted into the central cavity 204 of the barrel 202 through the upper opening 205. The central cavity 204 is then filled with a second predefined volume of a second fluid (e.g., a medication) through the upper opening 205. The second gasket 254 is then inserted into the central cavity 204 through the upper opening 205 such that the second fluid is stored between the first gasket 252 and the second gasket 254. The central cavity 204 is then filed with a third predefined volume of a third fluid (e.g., a second saline flush) and the end stopper 250 and the plunger 260 are then inserted into the central cavity 204 through the upper opening 205, as shown in FIG. 2.

In some implementations, the first gasket 252 is inserted into the central cavity 204 prior to filling the central cavity 204 with fluid. The second fluid, the second gasket 254, the third fluid, the end stopper 250, and the plunger 260 are then assembled as described above. In other words, the second chamber 220 and the third chamber 230 are prefilled with fluid. In such implementations, the tip 206 or a needle coupled to the tip (e.g., needle 172) is inserted into a vial of the first fluid. Actuation of the plunger 260 in the opposite direction of arrow A causes the first fluid to be drawn up into the first chamber 210 as a result of a pressure differential. In this manner, the first chamber 210, the second chamber 220, and/or the third chamber 230 can be prefilled (i.e., such that the fluid(s) are stored in the multi-chamber syringe 200).

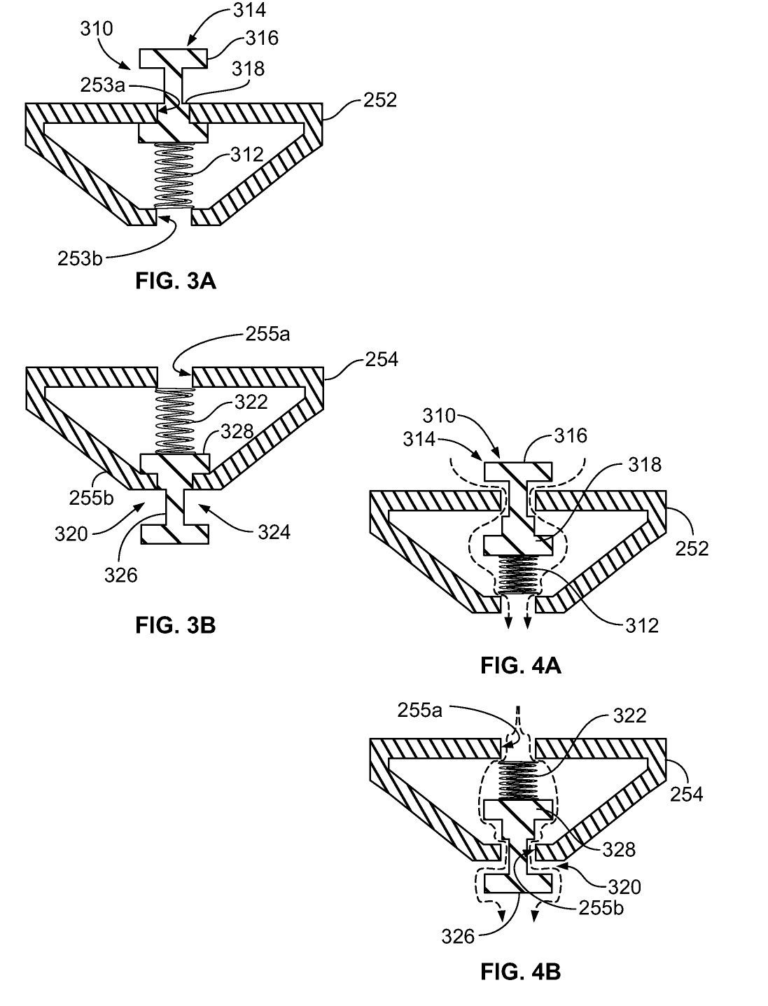

As described above, the first gasket 252 includes a first valve assembly and the second gasket 254 includes a second valve assembly. The valve assemblies are used to selectively permit fluid to flow through the gaskets (and thus between chambers) of the multi-chamber syringe 200. Referring to FIGS. 3A-4B, the first gasket 252 includes an exemplary first valve assembly 310 (FIGS. 3A and 4A) and the second gasket 254 includes an exemplary second valve assembly 320 (FIGS. 3B and 4B). The first gasket 252 includes an upper opening 253a and a lower opening 253b, and the second gasket 254 includes an upper opening 255a and a lower opening 255b.

As shown in FIGS. 3A and 4A, the exemplary first valve assembly 310 includes a spring 312 and a stopper 314. The stopper 314 extends through the upper opening 253a of the first gasket 252 and is partially disposed within the first gasket 252. The stopper 314 includes an upper portion 316 and a lower portion 318. As shown, the spring 312 generally urges the stopper 314 towards the upper opening 253a and causes the lower portion 318 of the stopper 314 to seal the upper opening 253a. When sufficient force (i.e., pressure) is exerted on the upper portion 316, the force overcomes the biasing of the spring 312 and moves the stopper 314 towards the lower opening 253b. Thus, the valve assembly 310 can be transitioned from a closed position (FIG. 3A) to an open position (FIG. 4A) to permit fluid to pass through the first gasket 252. For example, the force may come from the second gasket 254 contacting the first gasket 252 during operation as described above.

Referring to FIGS. 3B and 4B, the exemplary second valve assembly 320 of the second gasket 254 is the same as or similar to the first valve assembly 310 in that it includes a spring 322 and a stopper 324. The stopper 324 includes a lower portion 326 and an upper portion 328. The second valve assembly 320 differs from the first valve assembly 310 in that the stopper 324 is extends through the lower opening 255b, rather than the upper opening 255a. The second valve assembly 320 can move between a closed position (FIG. 3B) and an open position (FIG. 4B) in the same or similar manner as the first valve assembly 310 (e.g., when the second gasket 254 contacts the first gasket 252 during operation, as described above).

While the first and second gaskets 252, 254 have been shown and described as including first and second valve assemblies 310, 320 respectively, other valve assembly mechanisms and/or gaskets are possible for selectively permitting fluid to flow between chambers through the gaskets of the multi-chamber syringe 200.

As described above, the first alternative multi-chamber syringe (not shown, but including the barrel 102, the plunger 160, the end stopper 150, and the first gasket 152 of FIG. 1) can be configured for a saline-administer (“SA”) protocol. The first alternative multi-chamber syringe is the same or similar to the multi-chamber syringe 200 but does not include the second gasket 254 (i.e., the first alternative multi-chamber syringe only includes two chambers, rather than three). In such implementations, a medication is stored in the second chamber (which is defined by the space between the first gasket 152 and the end stopper 150), and a saline flush is stored in the first chamber (which is defined by the space between the tip 106 and the first gasket 152). In this configuration, actuation of the plunger 160 causes the two fluids to be sequentially dispensed through the tip 106: the first saline flush is dispensed first and the medication is dispensed second. The first saline flush clears the line (e.g. the central venous catheter 180) prior to the administration of the medication. The first chamber and/or the second chamber can be prefilled or filled prior to use in accordance with the principles described herein.

Advantageously, the multi-chamber syringes 100, 200 described herein can store a plurality of fluids (e.g., a saline flush and a medication) without the fluids mixing within the syringe. Mixing of the fluids may destroy the efficacy of a medication or create a mixture of fluids that is dangerous for the patient. Further, the multi-chamber syringes 100, 200 allow the plurality of fluids to be delivered sequentially using only a single connection to an access point on a central venous catheter (i.e., central venous catheter 180).

Additionally, the SASH and SASA protocols would require four conventional syringes (i.e., having one chamber), the SAS protocol would require three conventional syringes, and the SA protocol would require two conventional syringes. Advantageously, by using the multi-chamber syringes described herein, each protocol can be carried out with a single syringe. And because central line-associated bloodstream infections (“CLABSI”) are directly correlated with the number of syringes that are used, the use of the multi-chamber syringes for each protocol substantially decreases the likelihood of the patient contracting a CLABSI. Further still, prefilling one or more chambers of a multi-chamber syringe (e.g., with a flushing solution) reduces the amount of time needed to prepare each protocol.

While the present disclosure has been described with reference to one or more particular embodiments, those skilled in the art will recognize that many changes may be made thereto without departing from the spirit and scope of the present disclosure. Each of these embodiments and obvious variations thereof is contemplated as falling within the spirit and scope of the disclosure. It is also contemplated that additional embodiments according to aspects of the present disclosure may combine any number of features from any of the embodiments described herein.

what is claimed is:

1. A multi-chamber syringe for delivering a plurality of fluids to a central venous catheter, comprising:
a barrel including a plurality of chambers, a first one of the plurality of chambers having a predefined volume of a saline flush stored therein; and
a plunger, wherein actuation of the plunger causes the predefined volume of the saline flush to be dispensed from the barrel into the central venous catheter.

2. The multi-chamber syringe of claim 1, wherein a second one of the plurality of chambers has a predefined volume of at least one of a medication, a nutritional fluid, or a supplement fluid stored therein, wherein actuation of the plunger causes the predefined volume of medication, nutritional fluid, or supplement fluid to be dispensed into the central venous catheter subsequent to the saline flush.

3. The multi-chamber syringe of claim 2, wherein a third one of the plurality of chambers has a predefined volume of a second saline flush stored therein, wherein actuation of the plunger causes the predefined volume of the second saline flush to be dispensed into the central venous catheter subsequent to the medication, nutritional fluid, or supplement fluid and the saline flush.

4. The multi-chamber syringe of claim 3, wherein a fourth one of the plurality of chambers has a predefined volume of a heparin flush stored therein, wherein actuation of the plunger causes the predefined volume of the heparin flush to be dispensed into the central venous catheter subsequent to the second saline flush, the medication, nutritional fluid, or supplement fluid and the saline flush.

5. The multi-chamber syringe of claim 3, wherein a fourth one of the plurality of chambers has a predefined volume of an antibiotic flush stored therein, wherein actuation of the plunger causes the predefined volume of the antibiotic flush to be dispensed into the central venous catheter subsequent to the second saline flush, the medication, nutritional fluid, or supplement fluid and the saline flush.

6. A multi-chamber syringe for delivering a plurality of fluids to a central venous catheter, comprising:
a barrel including an upper opening, a central cavity, and a tip having a lower opening;
a gasket and an end stopper disposed within the central cavity such that (i) a first chamber of the central cavity is defined by a space between the tip and the gasket, and (ii) a second chamber of the central cavity is defined by a space between the gasket and the end stopper, the gasket having a valve assembly configured to move between a closed position and an open position, the first chamber having a predefined volume of flushing solution stored therein; and
a plunger slidably disposed within the central cavity such that the plunger is moveable with respect to the barrel;
wherein responsive to the valve assembly of the gasket being in the open position, actuation of the plunger causes at least a portion of the predefined volume of flushing solution stored in the first chamber to be dispensed through the lower opening of the tip.

7. The multi-chamber syringe of claim 1, wherein the flushing solution stored in the first chamber is at least one of a saline flush, a heparin flush, an antibiotic flush, or any combination thereof.

8. The multi-chamber syringe of claim 1, wherein the second chamber has a predefined volume of fluid stored therein such that actuation of the plunger causes the predefined volume of fluid stored in the second chamber to be dispensed through the lower opening of the tip.

9. The multi-chamber syringe of claim 8, wherein the fluid stored in the second chamber is at least one of a medication, a nutritional fluid, or a supplement fluid, or any combination thereof.

10. The multi-chamber syringe of claim 1, further comprising a second gasket having a second valve assembly and being disposed within the central cavity such that (i) the second chamber of the central cavity is defined by a space between the gasket and the second gasket, and (ii) a third chamber of the central cavity is defined by a space between the second gasket and the end stopper, the second valve assembly being configured to move between a closed position and an open position for permitting fluid communication between the third chamber and the second chamber.

11. The multi-chamber syringe of 10, wherein a predefined volume of a second flushing solution is stored in the third chamber of the central cavity.

12. The multi-chamber syringe of claim 11, wherein the second flushing solution stored in the third chamber is at least one of a saline flush, a heparin flush, an antibiotic flush, or any combination thereof.

13. The multi-chamber syringe of claim 10, further comprising a third gasket having a third valve assembly and being disposed within the central cavity such that (i) the third chamber of the central cavity is defined by a space between the second gasket and the third gasket, and (ii) a fourth chamber of the central cavity is defined by a space between the third gasket and the end stopper, the third valve assembly being configured to move between a closed position and an open position for permitting fluid communication between the fourth chamber and the third chamber.

14. The multi-chamber syringe of claim 13, wherein a predefined volume of a third flushing solution is stored in the fourth chamber of the central cavity.

15. The multi-chamber syringe of claim 14, wherein (i) the flushing solution stored in the first chamber is a saline solution, (ii) the fluid stored in the second chamber is a medication, (iii) the second flushing solution stored in the third chamber is a saline solution, and (iv) the third flushing solution stored in the fourth chamber is a heparin flush or an antibiotic flush.

16. A method for delivering a plurality of fluids to a central venous catheter, the method comprising:
providing a multi-chamber syringe including:
a barrel having an upper opening, a central cavity, and a tip having a lower opening;
a gasket having a valve assembly and an end stopper disposed within the central cavity such that (i) a first chamber of the central cavity is defined by a space between the gasket and the end stopper, and (ii) a second chamber of the central cavity is defined by a space between the tip and the gasket, the first chamber having a predefined volume of flushing solution stored therein; and
a plunger slidably disposed within the central cavity;
removing the plunger and the end stopper from the barrel and dispensing a predefined volume of medication into the second chamber;
slidably engaging the end stopper and plunger with the central cavity of the barrel;
coupling the tip of the multi-chamber syringe to an access port of the central venous catheter;
moving the plunger from a first position to a second position such that the predefined volume of medication stored in the second chamber is dispensed through the lower opening of the tip; and
moving the plunger from the second position to a third position such that the predefined volume of flushing solution stored in the first chamber is dispensed through the lower opening of the tip.

17. The method of claim 11, wherein the flushing solution stored in the first chamber is at least one of a saline flush, a heparin solution, an antibiotic flush, or any combination thereof.
